(19) United States
(12) Patent Application Publication
(10) Pub. No.: US 2023/0390176 A1
(43) Pub. Date: Dec. 7, 2023

(54) Cosmetic Skin and Medical Cream

(71) Applicant: Protecta-Skincare, Inc., Los Angeles, CA (US)

(72) Inventor: Robert H. Schiestl, Los Angeles, CA (US)

(21) Appl. No.: 18/453,324

(22) Filed: Aug. 22, 2023

Related U.S. Application Data
(63) Continuation-in-part of application No. 17/398,960, filed on Aug. 10, 2021.

(51) Int. Cl. A61K 8/49; A61K 31/4709; A61K 9/06; A61Q 19/00

(52) U.S. Cl. CPC A61K 8/4946; A61K 31/4709; A61K 9/06; A61Q 19/00

(57) ABSTRACT

A medicated cosmetic skin cream containing YEL002 as the active ingredient and a combination of ginger oil, aloe vera gel, bees wax, cacao butter, avocado oil, and a cosmetic base can be used to treat many skin conditions. The skin cream is effective at treating keratosis, psoriasis, inflammation of the skin, skin wrinkles, discoloration, scarring, sagging skin, acne, precancerous skin lesions, sunburn, arthritis, incisions during surgery, whiplash syndrome, abscesses, skin pain, warts, broken toes or fingers, burn blisters, burn inflammation, burn pain, and Hidradenitis Suppurativa.

### CROSS-REFERENCE TO RELATED APPLICATIONS

This application is a continuation-in-part of U.S. application Ser. No. 17/398,960 titled “Cosmetic Skin Cream and Medicine” and filed Aug. 10, 2021, of which the entire contents therefore are hereby incorporated by reference into the present disclosure.

### BACKGROUND OF THE INVENTION

The present disclosure relates generally to a novel skin cream and medicine composition and more particularly to a skin cream containing the compound YEL002 and skin-beneficial substances.

In general, exposure to UV, ionizing radiation, radioactive iodine, environmental pollution, cigarette smoke, and chemical carcinogenic agents exert severe stress on the skin, often times leading to accelerated aging of the skin such as the appearance of wrinkles, sagging skin, discoloration, inflammation, itching, and other cosmetically undesirable effects, as well as more health-related issues such as skin cancer.

Additionally, many people suffer from skin-related issues, including psoriasis, keratosis, acne, eczema, wounds, incisions, precancerous skin lesions, inflammation, skin pains and pains under the skin, as well as deep tissue diseases. Additionally, almost all surgeries make incisions in the skin which must heal.

Warts: Common warts are small, grainy skin growths that occur most often on your fingers or hands. Rough to the touch, common warts also often feature a pattern of tiny black dots, which are small, clotted blood vessels and may itch and bleed, especially when scratched. Common warts are caused by a virus and are transmitted by touch. Warts are very common, especially among children and teenagers. Various studies have shown that up to 33% of children and teenagers have warts. They are estimated to be much less common in other age groups, affecting only about 3 to 5% of adults. This translates to about 700 million people worldwide. Current treatments include salicylic acid, freezing or laser treatments that might leave scars. Current market size is about $1 trillion. It would be great to have a treatment of warts that does not leave scars and maybe even treats past or current scars.

Broken toes and fingers: A broken toe is a common injury that's most often caused by dropping something on a foot or stubbing a toe. Usually, treating a broken toe involves taping it to the next toe. But if the fracture is severe—especially if it's in the big toe—proper healing might require a cast or even surgery to heal well. Most broken toes heal well, usually within 4 to 6 weeks. Sometimes, however, a broken toe can get infected. Also, the break might increase the risk of getting arthritis in that toe in the future. It would be great if one could heal broken toes and fingers faster and if one could also treat the resulting arthritis. The worldwide market size is more than $5 billion.

Burns: Current treatment includes cooling the burn with cool or lukewarm running water for 20 minutes as soon as possible after the injury. Never use ice, iced water, or any creams or greasy substances like butter. Keep yourself or the person warm. Use a blanket or layers of clothing but avoid putting them on the injured area. It would be great if one had a treatment of burns that also gets rid of current and past burn scars. Prevalence, about 10 million people, 33,000 per day. Annual worldwide market size is $2.6 billion.

Hidradenitis Suppurativa: Also called acne inversa, HS can look like pimples and acne cysts. It's not acne, though. Still, when HS first appears, some people think that they have pimples in their armpit, on their inner thighs, or in their groin area. Without treatment for HS, this skin condition can worsen, is inflamed and very painful. Abscesses, which are pockets of pus, can develop. When an abscess breaks open, blood and pus leak out. As the abscess heals, a scar usually develops. It would be great to have a treatment for Hidradenitis Suppurativa, that does not result in scars and maybe past scars can be treated too. The current annual market size of treatment is about $900 million.

The compound YEL002, shown below, has been discovered to be effective in mitigating tissue damage induced by an agent:

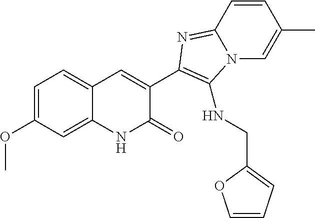 [SMILES: COc1ccc2cc(-c3nc4ccc(C)cn4c3NCc3ccco3)c(=O)[nH]c2c1]

The effectiveness and manufacture of YEL002 was disclosed in U.S. Pat. No. 9,045,474, granted on Jun. 2, 2015, and titled COMPOUNDS AND COMPOSITIONS FOR MITIGATING TISSUE DAMAGE AND LETHALITY, the entirety of which is incorporated herein by reference.

In order to overcome the skin problems described above, there are many cosmetic compositions on the market for use on the skin. However, there is a need for skin treatments that are economical, effective, and simple to apply, such as in a skin cream, that are capable of smoothing and firming the skin, while at the same time treating skin diseases such as psoriasis and keratosis and protecting against skin damage resulting from UV and pollutant exposure, inflammation, wounds, incisions, as well as other medical diseases and pains on and below the skin.

The embodiments described below address the above-mentioned problems and needs.

### BRIEF SUMMARY

In accordance with one embodiment of the present disclosure, there is contemplated a skin cream comprising YEL002. In certain embodiments, the skin cream may further include at least one oil. More generally, the skin cream may contain between 0.2-5% YEL002, between 0.5-4.0% ginger oil, between 3-30% aloe vera gel, between 1-10% bees wax, between 3-40% cacao butter, between 50-90% avocado oil, and between 0.5%-3.0% of cosmetic base (COSPHADERM®). In one specific embodiment, the skin cream may contain approximately 1% YEL002, approximately 2% ginger oil, approximately 9% aloe vera gel, approximately 2.5% bees wax, approximately 7.5% cacao butter, approximately 77% avocado oil, and approximately 1.4% cosmetic base. Other embodiments may further include between 0.005-0.5% human recombinant albumin.

Another embodiment of the present disclosure contemplates a method of manufacturing a skin cream. In this embodiment, the skin cream is made by disposing a first container containing avocado oil in a water bath. The water bath is heated to approximately 90° C. Next, beeswax and cacao butter is dissolved into the first container. Next, aloe vera gel is introduced into a second container. Then, the contents of the first container are slowly poured into the second container, while the second container is mixed with a fast mixer to obtain a homogenous emulsion. Finally, YEL002, ginger oil and cosmetic base is mixed into the second container until a homogenous emulsion is formed.

Yet another embodiment of the present disclosure contemplates a method of treating a skin condition by applying a skin cream containing YEL002 to the skin condition. Examples of skin conditions that may be treated under this embodiment include keratosis, psoriasis, inflammation of the skin, skin wrinkles, discoloration, lipomas, scarring, sagging skin, acne, precancerous skin lesions, incisions after surgery, and sunburn. In this method, the skin cream may include at least one oil. For example, the skin cream may contain between 0.2-5% YEL002, between 0.5-4% ginger oil, between 0.5 to 3% cosmetic base, between 3-30% aloe vera gel, between 1-10% bees wax, between 3-40% cacao butter, and between 50-90% avocado oil. In a specific embodiment of the method, the skin cream may contain approximately 1% YEL002 approximately 2% ginger oil, approximately 1.4% cosmetic base, approximately 9% aloe vera gel, approximately 2.5% bees wax, approximately 7.5% cacao butter, and approximately 77% avocado oil. The skin cream may also be used to provide pain relief to a user having pain on the skin or up to about 1.5 inches below the skin. This pain relief is achieved by applying a skin cream containing YEL002 to the location of pain.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

A new combination of ingredients, including a radiation mitigator, namely, YEL002, results in a cosmetic cream that provides protection against many types of skin damage. The cosmetic cream further acts to heal skin and under-the-skin diseases and pains. YEL002, shown below, is one active ingredient in the skin cream.

The skin cream may include other cosmetic components known in the field. In a particular embodiment, the skin cream is made up of a combination of YEL002, ginger oil, aloe vera gel, bees wax, cacao butter, avocado oil, and a cosmetic base. For example, one embodiment of the skin cream contains between 0.2-5% YEL002, between 0.5-4% ginger oil, between 0.5-3% cosmetic base, between 3-30% aloe vera gel, between 1-10% bees wax, between 3-40% cacao butter, and between 50-90% avocado oil. More particularly, one formulation contains about 1% YEL002, about 2% ginger oil, 1.4% cosmetic base, about 9% aloe vera gel, about 2.5% bees wax, about 7.5% cacao butter, and about 77% avocado oil. Other embodiments include 0.005-0.5% human recombinant albumin. In a preferred embodiment, the cosmetic base may be COSPHADERM® (a commercially-available cosmetic base containing 1,2-hexanediol, phenylpropanol, xanthan gum, and lecithin).

Each of the cosmetic components disclosed above contribute to the improved properties of the cosmetic skin cream and medical cream described herein and is present in a quantity sufficient to increase the smoothness and firmness of the skin to which the skin cream is applied, as well as providing protection against environmental pollutants and UV exposure. Further, application of the skin cream makes the skin glow and is anti-inflammatory and anti-aging. Additionally, these components in the skin cream are useful in treating skin conditions, such as psoriasis, keratosis, eczema, acne and the like. However, it is contemplated that other suitable oils or beneficial substances may be utilized, and other percentages of the components may be utilized. Additionally, the skin cream may further include other cosmetic components to add additional benefits to the skin cream, for example, but not limited to, emulsifiers, antioxidants, preservatives, solvents, thickeners, fragrances, colorants, human recombinant albumin, and the like. While it is common for skin creams within the art to be comprised of 70-80% aqueous solutions, the novel skin cream disclosed herein is made up of approximately 75% oils.

The cosmetic skin and medical cream described herein provides numerous benefits to the skin of the user, but notably the cream makes the user's skin smoother and firmer. The skin cream described herein also has anti-inflammatory and anti-aging (human keratinocytes live 15% longer with the application of the active ingredient YEL002) properties and prevents wrinkles with continued use. The cream also beautifies the skin and makes it glow. Furthermore, the skin cream described herein has antibacterial and antifungal properties which serves to reduce the load on the immune system. In addition, rather than suppressing it, application of the cream redirects the immune system to be anti-inflammatory in relieving the inflammation in joints caused by arthritis. Also, the skin cream is effective in healing the pain and the skin wounds and incisions from surgery each at a rate 7× faster than without the cream.

With use, the skin cream described herein has been shown and/or is believed to protect the skin against ionizing radiation, UV, chemical carcinogens, chemical warfare agents, radioactive iodine, and cigarette smoke. Notably, the skin cream described herein has also been shown to heal psoriasis, keratosis, eczema, inflammation, of the face and toes, acne, precancerous skin lesions, sunburn, scars, discolored skin patches, to stop itching from insect and tick bites, and help prevent skin cancer. Unlike the use of cyclosporine to treat psoriasis, the skin cream as discussed above does not have immuno-suppressant effects. Also, the skin cream does not have the deleterious effect of disrupting DNA synthesis as occurs from use of the chemotherapy drug fluorouracil to treat keratosis. In particular, initial results with use of the cream described herein show improvement for ten patients from keratosis, three patients from psoriasis, two patients from eczema, one patient from inflammation of the face, three patients from inflammation of the toes, five patients from skin wrinkling, one patient from sunburn, two patients from scarring, three patients from discolored patches, one patient from precancerous lesions, and three patients from itching due to insect and tick bites. Additionally, more than 600 people saw their skin beautified and glowing from use of the skin cream. Some saw their hair look more shiny and healthy when using the cream on their hair. As such, the skin cream has applications throughout the human and animal body.

The skin cream also has deep tissue effects. It is well known that NSAIDS like ibuprofen are anti-inflammatory and analgesic. The skin cream disclosed herein is highly anti-inflammatory and thus also acts as a natural pain killer. It has been found that with use, the skin cream heals skin pains and pains up to about 1.5 inches below the skin (which is approximately 95% of all pains) after 1-3 weeks by applying the skin cream 1-5 times a day in a very thick layer, which is absorbed into the skin in about 1-20 minutes. Also, application of the skin cream is effective in eliminating pain at a rate 7× faster than without the cream. It has been observed that neck pains, knee pains, back pains, wrist and hand pains, inflamed tendons, rib and leg pains have been healed by such use of the skin cream. It has also been noted to heal whiplash syndrome, cysts, abscesses, arthritis, and lipomas. In particular, early results show relief from pain in the neck for two patients, in the knee for two patients, in the back for one patient, in the wrist and hands for three patients, in inflamed tendons for one patient, in the leg for four patients, along with relief from whiplash syndrome for one patient, cysts and abscess each for one patient, arthritis for three patients, and lipomas for two patients.

While the skin cream described herein may be formulated in suitable manners as is known in the art, it has been found to be effective to manufacture the skin cream by mixing the oils together in a heated water bath, and to then mix the aloe vera gel with a fast mixer and slowly add the oils to generate a smooth emulsion, and finally mix in the remaining ingredients.

In the treatment of warts, after continual treatment of warts 4-5 times a day with the skin cream the warts dry up and fall out or can be removed including the root, after which a little hole appears in the skin which after continued treatment disappears.

After treatment of broken toes or fingers 4-5 times a day all around the broken area the break heals much faster. This is because the cream is a pain killer, anti-inflammatory, and wound-healer.

The cream gets rid of the burn blisters, burn inflammation, and pain. The resulting burn scar is smaller than without the cream and the scar disappears upon further treatment.

Treatment of all affected skin areas of a Hidradenitis Suppurativa patient with the cream gets rid of the pain, the swelling and the inflammation as well as the pus accumulation the cysts, abscesses etc. Past treatment of scars resulting from the disease leads to disappearance of such scars.

The above description is given by way of example, and not limitation. Given the above disclosure, one skilled in the art could devise variations that are within the scope and spirit of the invention disclosed herein, including adjusting the proportion of the components within the composition. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of treating a skin condition, said method comprising applying a skin cream containing YEL002 to the skin condition.

2. The method of claim 1, wherein the skin condition is keratosis.

3. The method of claim 1, wherein the skin condition is psoriasis.

4. The method of claim 1, wherein the skin condition is inflammation of the skin.

5. The method of claim 1, wherein the skin condition is skin wrinkles.

6. The method of claim 1, wherein the skin condition is discoloration.

7. The method of claim 1, wherein the skin condition is scarring.

8. The method of claim 1, wherein the skin condition is sagging skin.

9. The method of claim 1, wherein the skin condition is acne.

10. The method of claim 1, wherein the skin condition is a precancerous skin lesion.

11. The method of claim 1, wherein the skin condition is sunburn.

12. The method of claim 1, wherein the skin condition is arthritis.

13. The method of claim 1, whereas the skin condition is skin incisions during surgery.

14. The method of claim 1, whereas the skin condition is whiplash syndrome.

15. The method of claim 1, whereas the skin condition is abscess.

16. The method of claim 1, whereas the skin condition is skin pain.

17. The method of claim 1, wherein the skin condition is warts.

18. The method of claim 1, wherein the skin condition is broken toes or fingers.

19. The method of claim 1, wherein the skin condition is burn blisters, burn inflammation, and pain caused by burns.

20. The method of claim 1, wherein the skin condition is Hidradenitis Suppurativa.
